(19) United States
(12) Patent Application Publication
(10) Pub. No.: US 2017/0189706 A1
(43) Pub. Date: Jul. 6, 2017

(54) TARGETED DRUG DELIVERY DEVICE FOR ANTI-TUMOR MAGNETIC NANOPARTICLE DRUGS

(71) Applicant: Guangzhou Yidai Pharmaceutical Co., Ltd., Guangzhou City (CN)

(72) Inventor: HUA WANG, GUANGZHOU CITY (CN)

(21) Appl. No.: 15/306,114

(22) PCT Filed: Dec. 30, 2014
(86) PCT No.: PCT/CN2014/095544

§ 371 Date: Dec. 23, 2016

(30) Foreign Application Priority Data
Mar. 5, 2014 (CN) 201410078803.9

(51) Int. Cl. A61N 2/00; A61N 2/06; A61N 1/40; A61N 2/02

(52) U.S. Cl. CPC A61N 2/00; A61N 2/004; A61N 2/02; A61N 2/06; A61N 1/406; B82Y 5/00

(57) ABSTRACT

Provided is a targeted drug delivery device for antitumor magnetic nanoparticle drugs including a transfusion container for accommodating antitumor magnetic nanoparticle drugs and a drug solution conveying device connected to the transfusion container and used for conveying the antitumor magnetic nanoparticle drugs. A magnetic field generating device capable of producing magnetic adsorption to the antitumor magnetic nanoparticle drug and a bioelectric sensor for sensing the response of living tissues in a tumor region to magnetic stimulation are arranged at an external position of the human body corresponding to the tumor region. The targeted drug delivery device is provided with a control unit for receiving and analyzing signals of the bioelectric sensor and providing a master control on the required magnetic field intensity, transfusion speed and liquid flow according to the signals of the bioelectric sensor.

### CROSS-REFERENCE TO RELATED APPLICATIONS

This application claims priority to PCT Application No. PCT/CN2014/095544, having a filing date of Dec. 30, 2014, based off of Chinese application No. 201410078803.9 having a filing date of Mar. 5, 2014, the entire contents of which are hereby incorporated by reference.

### FIELD OF TECHNOLOGY

The following relates to the technical field of delivery device product, particularly refers to a targeted drug delivery device for antitumor magnetic nanoparticle drugs.

### BACKGROUND

Nanoparticle (also called as ultrafine particle) is a type of particle having a particle size ranging between 1 to 100 nm, and it falls in the value range of particle size of colloid particle. As a group consisting of a small number of atoms or molecules, nanoparticles are in a transition region between atom clusters and macroscopical objects, and are between the microscopic system and macroscopical system, thus they are neither in a typical microscopic system, nor in a typical macroscopic system, and some new physical and chemical characteristics of the nanoparticles can be anticipated. The structural features of the nanoparticle, which make the nanoparticle differ from the macroscopic objects, are that the nanoparticle has a high specific surface area and the surface atoms, without long-range order nor short-range order, form an amorphous layer. It can be considered that the surface atoms of the nanoparticles are more nearly gaseous, while the atoms in the inner of the particles are possibly arranged in order. Even so, some deformations of the inner structure may occur by high Gilibis pressure generated in the inner, due to the small particle sizes and big surface curvatures.

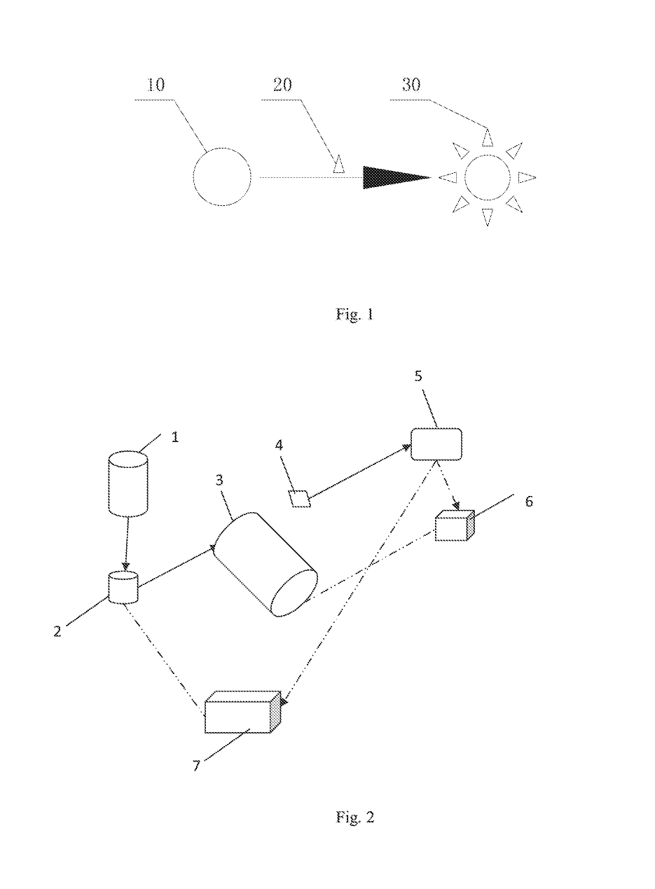

The nanomaterial has been widely used in medicine and bioengineering. A targeted drug, taking the magnetic nanomaterial as the drug carrier, has been successfully developed, and it is called a “biological missile”, i.e., the drugs are carried on the surface of proteins coated with magnetic nanoparticles. As shown in FIG. 1, antitumor drugs 20 such as pingyangmycin are carried on the surface of a magnetic nanoparticle 10, and a magnetic nanoparticle antitumor drug 30 is obtained through physical or covalent bonds therebetween. The magnetic nanoparticle antitumor drugs 20 enter pathological tissues through blood vessel injection or tumor intracavity injection, in order to decrease the side-effects caused by the drugs on the liver, spleen, kidney, etc. However, those drugs entering the nidus regions through tumor intracavity injection or blood vessel perfusion can be reabsorbed and introduced into the blood circulation. Especially regarding those niduses having rich vascular circulation, such as hemangiosarcoma, malignant melanoma, arterious malformations (cirsoid angioma) and large venous malformations, the blood in the nidus regions flows fast, thus the drugs stay for a short time in the tissues in the nidus regions after they enter the nidus regions. As a result, the drug concentration is low and medicinal effect is poor, which affects the curative effect for tumor patients. In addition, the drugs entering the blood circulation will damage tissues of organs of human beings. Therefore, it is extremely urgent to develop a targeted device capable of controlling the antitumor magnetic nanoparticles drugs to concentrate in the tumor region.

### SUMMARY

An aspect relates to a targeted drug delivery device for antitumor magnetic nanoparticle drugs. Such targeted drug delivery device is capable of decreasing the flow of the magnetic nanoparticle drugs, and controlling the drugs to concentrate in the tumor regions, so as to achieve a high drug concentration in the tumor region and an excellent medicinal effect, which is advantageous for the recovery of the tumor patients and is also helpful to reduce the unnecessary harm on the human tissue organisms caused by drugs.

In order to achieve this aspect, embodiments of the present invention provides the following technical solutions:
a targeted drug delivery device for antitumor magnetic nanoparticle drugs of the present invention comprises a transfusion container for accommodating antitumor magnetic nanoparticle drugs and a drug solution conveying device connected to the transfusion container and used for conveying the antitumor magnetic nanoparticle drugs; a magnetic field generating device capable of producing magnetic adsorption to the antitumor magnetic nanoparticle drugs and a bioelectric sensor for sensing the response of living tissues in a tumor region to magnetic stimulation are arranged at an external position of the human body corresponding to the tumor region; and a control unit is further configured for receiving and analyzing signals of the bioelectric sensor and providing a master control on the required magnetic field intensity, transfusion speed and liquid flow, according to the signals of the bioelectric sensor; a magnetic field control device for controlling the magnetic field intensity is connected to the magnetic field generating device, a flow rate controller is connected to the drug solution conveying device to control its flow rate; the control unit is connected to the bioelectric sensor, the magnetic field control device and the flow rate controller respectively.

As a further improvement for the above technology, the bioelectric sensor comprises a surface electrode or a needle electrode arranged on the surface of treated region corresponding to the tumor region, a bioelectric amplifier and a signal processing system, and the surface electrode or the needle electrode is used to detect the bioelectric response of the tissues in the treated region to the stimulation of the magnetic force and the magnetic antitumor nano-drugs, and the bioelectric response will be conveyed to the control unit after a bioelectric amplification through the bioelectric amplifier and a signal analysis and processing through the signal processing system.

In embodiments of the present invention, the magnetic field generating device can be provided in the following two forms:

Firstly, the magnetic field generating device is in a form of alternating current coils which generate a rotating magnetic field, and it is secured at an external position of the human body corresponding to the tumor region. A magnetic field control device, connected to the magnetic field generating device, can be used to effectively control the magnetic field intensity and the magnetic field rotating speed.

Secondly, the magnetic field generating device is in a form of a number of magnetic materials adhering to an external position of the human body corresponding to the tumor region, by medical dressings or adhesive tapes; the magnetic materials may be magnets, and the magnetic force of the magnetic device can be controlled by adjusting the amount of magnets; besides, medical breathable materials may be filled between the magnetic materials and the medical dressings or adhesive tapes, to achieve better air permeability.

Compared with the known art, embodiments of the present invention has following advantages:
1) the magnetic antitumor nano-drugs are mainly concentrated in the tumor region by the magnetic field generating device arranged at an external position of the human body corresponding to the tumor region, whereby the concentration of the antitumor drugs in the tumor region is increased and the antitumor drugs reabsorbed and introduced into the blood circulation are reduced, so as to achieve a targeted drug delivery, better medical effects and reduced harm to other human organisms caused by drugs;
2) the magnetic field control device can be provided to control the intensity of the magnetic field, thus the magnetic field intensity would be increased if the tumors are located far from the body surface, on the contrary, the field intensity would be decreased; it is well-controlled and good for the health of human beings;
3) a bioelectric sensor is further provided to receive the bioelectric response of tissues in the treated region to the stimulation of magnetic field force and magnetic antitumor nano-drugs, and to evaluate the intensity and the influence scope for the tissues in the tumor region, and then adjust the magnetic field intensity and drug dose, which is helpful to increase the accuracy of the treatment, decrease the complication and improve the medical safety;
4) embodiments of the present invention is especially suitable for those benign or malignant tumors having rich vascular circulation, as the magnetic antitumor nano-drugs can stay in the nidus regions for a longer time by magnetic force, which brings a new therapeutic hope for those patients suffering from hemangiosarcoma, malignant melanoma, arterious malformations (cirsoid angioma) and large venous malformations, on which the current therapeutic effect is unsatisfactory.

### BRIEF DESCRIPTION

Some of the embodiments will be described in detail, with reference to the following figures, wherein like designations denote like members, wherein:

FIG. 1 is a schematic diagram showing the binding process of magnetic nanoparticles and antitumor drugs;

FIG. 2 is a structural schematic diagram of the targeted drug delivery device for magnetic nanoparticle antitumor drugs of embodiments of the present invention;

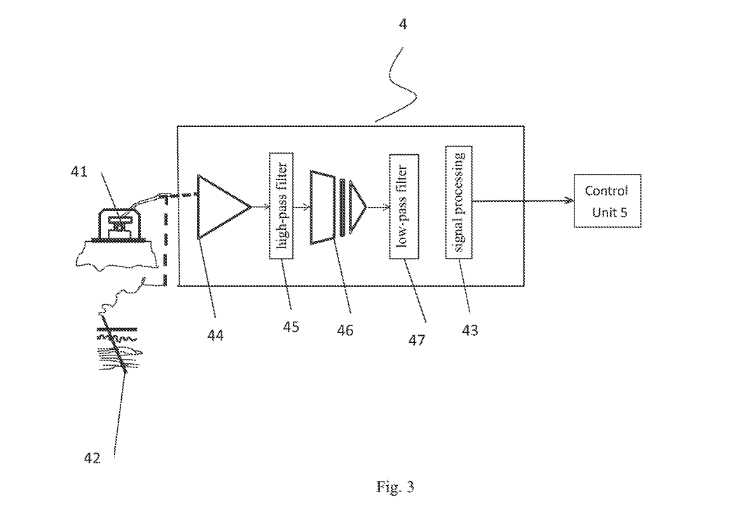

FIG. 3 is a schematic diagram showing the structure and connection of the bioelectric sensor according to Example 1;

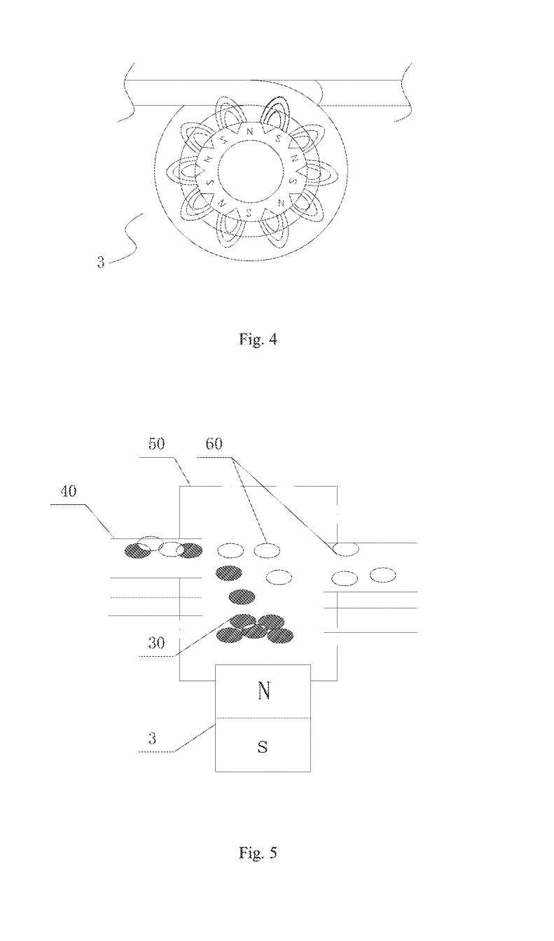

FIG. 4 is a structural schematic diagram of the magnetic field generating device according to Example 1;

FIG. 5 illustrates the therapy principle of the magnetic antitumor nano-drugs;

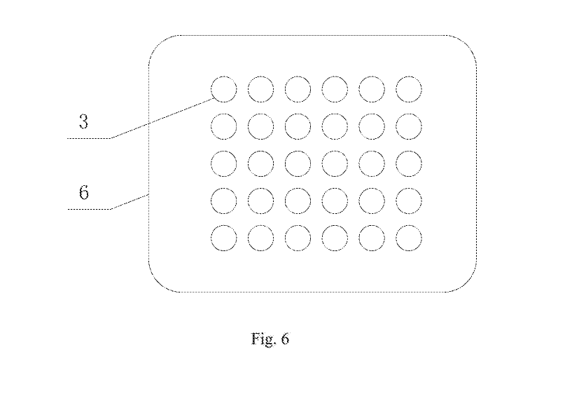

FIG. 6 is a structural schematic diagram of the magnetic field generating device according to Example 2.

### DETAILED DESCRIPTION

### EXAMPLE 1

As shown in FIG. 2, a targeted drug delivery device for antitumor magnetic nanoparticle drugs of embodiments of the present invention comprises a transfusion container 1 for accommodating antitumor magnetic nanoparticle drugs; and a drug solution conveying device 2 connected to the transfusion container 1 and used for conveying the antitumor magnetic nanoparticle drug. A magnetic field generating device 3 capable of producing magnetic adsorption to the antitumor magnetic nanoparticle drugs and a bioelectric sensor 4 for sensing the response of living tissues in a tumor region to magnetic stimulation are arranged at external positions of the human body corresponding to the tumor region. A control unit 5 is further provided for receiving and analyzing signals of the bioelectric sensor 4 and providing a master control on the required magnetic field intensity, transfusion speed and liquid flow according to the signals of the bioelectric sensor 4. A magnetic field control device 6 is connected to the magnetic field generating device 3 to control the magnetic field intensity. A flow rate controller 7 is connected to the drug solution conveying device 3 to control the flow rate of the drug solution. The control unit 5 is connected to the bioelectric sensor 4, the magnetic field control device 6 and the flow rate controller 7 respectively.

As shown in FIG. 3, the bioelectric sensor 4 comprises a surface electrode 41 or a needle electrode 42 arranged on the surface of the treated region corresponding to the tumor region, a bioelectric amplifier and a signal processing system 43. The bioelectric amplifier comprises a front amplifier 44, a high-pass filter 45, an isolation amplifier 46 and a low-pass filter 47 connected between each other. The bioelectric sensor 4 receives the bioelectric response of the tissues in the treated region to the stimulation of magnetic field force and magnetic antitumor nano-drugs, by the surface electrode 41 or needle electrode 42. The bioelectric response will be conveyed to the control unit 5 after a bioelectric amplification by the bioelectric amplifier and a signal analysis and processing by the signal processing system. The magnetic field intensity and the drug conveying speed are further adjusted by the control unit 5 according to the bioelectric signal strength and range in the treated region, and such control based on real-time feedback to the magnetic field control device 6 and control unit 5, according to the bioelectric signal of tissues in treated region in response to the stimulation of magnetic force and drugs, ensures a adjustment with a higher accuracy and improved safety performance.

As shown in FIG. 4, the magnetic generating device 3 is in a form of alternating current coils generating a rotating magnetic field, and it is secured at an external position of the body surface corresponding to the tumor region, by using the fasteners (no shown in figures). A magnetic control unit 6 is connected to the magnetic field generating device 3 to effectively control the intensity and rotating speed of the magnetic field. The magnetic control device 6 can adjust the magnetic field intensity of the magnetic field generating device 3 in real time, according to the distance between the tumor and the body surface, and the magnetic control device is conveniently used and well controlled. The magnetic field generating device 3 may be designed into corresponding shapes according to the shape of the treated region. For example, it may be jaw-shaped for the jaw tumor, cap-shaped for the brain tumor, and cannular for the tumors in thyroid gland of the neck, limbs, chest and abdomen.

The principle of the Example can be seen in FIG. 5, the magnetic nanoparticle antitumor drugs 30 flow from the blood vessels 40 into the tumor region 50, and they will be controlled in the tumor region 50 under the absorption of magnetic field generating device 3, whereas those cells 60 unlabelled by the magnetic nanoparticles in the tumor region 50 will continue to flow along the blood vessels 40.

### EXAMPLE 2

This Example is similar with Example 1, however they differ in that the magnetic generating device 3 is a number of magnetic materials adhering to an external position of the human body corresponding to the tumor region, by medical dressings or adhesive tapes 8, as shown in FIG. 6. The magnetic materials are magnets, and the magnetic force of the magnetic device can be controlled by adjusting the amount of the magnets. In order to make the magnetic materials and the profile of the treated region fit closely, and to improve the air permeability and flexibility, medical breathable materials are filled between the magnetic materials or between the magnetic materials and adhesive tapes.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1-6. (canceled)

7. A targeted drug delivery device for antitumor magnetic nanoparticle drugs, comprising a transfusion container for accommodating antitumor magnetic nanoparticle drugs and a drug solution conveying device connected to the transfusion container and used for conveying the antitumor magnetic nanoparticle drugs, wherein
a magnetic field generating device capable of producing magnetic adsorption to the antitumor magnetic nanoparticle drugs and a bioelectric sensor for sensing the response of living tissues in a tumor region to magnetic stimulation are arranged at an external position of the human body corresponding to the tumor region;
a control unit is further provided for receiving and analyzing signals of the bioelectric sensor and providing a master control for a required magnetic field intensity, transfusion speed and liquid flow according to signals of the bioelectric sensor;
a magnetic field control device for controlling the magnetic field intensity is connected to the magnetic field generating device;
a flow rate controller is connected to the drug solution conveying device to control its flow rate; and
the control unit is connected to the bioelectric sensor, the magnetic field control device and the flow rate controller respectively.

8. The targeted drug delivery device for antitumor magnetic nanoparticle drugs according to claim 7, wherein the bioelectric sensor comprises a surface electrode or a needle electrode arranged on a surface of the treated region corresponding to the tumor region, a bioelectric amplifier and a signal processing system; and the surface electrode or the needle electrode is used to detect a bioelectric response of tissues in the treated region to the stimulation of the magnetic force and magnetic antitumor nano-drugs, and the bioelectric response will be conveyed to the control unit after a bioelectric amplification through the bioelectric amplifier and a signal analysis and processing through the signal processing system.

9. The targeted drug delivery device for antitumor magnetic nanoparticle drugs according to claim 7, wherein the magnetic field generating device is in a form of alternating current coils which generating a rotating magnetic field, and is secured at an external position of the human body corresponding to the tumor region.

10. The targeted drug delivery device for antitumor magnetic nanoparticle drugs according to claim 8, wherein the magnetic field generating device is in a form of alternating current coils which generating a rotating magnetic field, and is secured at an external position of the human body corresponding to the tumor region.

11. The targeted drug delivery device for antitumor magnetic nanoparticle drugs according to claim 7, wherein the magnetic field generating device is in a form of a number of magnetic materials adhered at an external position corresponding to the tumor region by medical dressings or adhesive tapes.

12. The targeted drug delivery device for antitumor magnetic nanoparticle drugs according to claim 11, wherein the magnetic materials are magnets.

13. The targeted drug delivery device for antitumor magnetic nanoparticle drugs according to claim 12, wherein medical breathable materials are filled between the magnetic materials and the medical dressings or the adhesive tapes.

14. The targeted drug delivery device for antitumor magnetic nanoparticle drugs according to claim 8, wherein the magnetic field generating device is in a form of a number of magnetic materials adhered at an external position corresponding to the tumor region, by medical dressings or adhesive tapes.

15. The targeted drug delivery device for antitumor magnetic nanoparticle drugs according to claim 14, wherein the magnetic materials are magnets.

16. The targeted drug delivery device for antitumor magnetic nanoparticle drugs according to claim 15, wherein medical breathable materials are filled between the magnetic materials and the medical dressings or the adhesive tapes.
